(19) United States
(12) Patent Application Publication
(10) Pub. No.: US 2011/0014473 A1
(43) Pub. Date: Jan. 20, 2011

(54) POLYMER-COATED NANOPARTICLES

(76) Inventors: Jackie Y. Ying, Singapore (SG); Nandanan ERATHODIYIL, Singapore (SG); Nikhil R. JANA, Singapore (SG)

Correspondence Address: WOLF GREENFIELD & SACKS, P.C., 600 ATLANTIC AVENUE, BOSTON, MA, 02210-2206, US

(21) Appl. No.: 12/449,227

(22) PCT Filed: Jan. 31, 2007
(86) PCT No.: PCT/US2007/002536

§ 371 Date: Aug. 9, 2010

(51) Int. Cl. B32B 27/00; B05D 3/00; C07K 2/00

(52) U.S. Cl. 428/407; 427/299; 530/350; 977/773; 977/774

(57) ABSTRACT

Polymers for coating nanoparticles (e.g., colloid nanoparticles and quantum dots) and methods associated therewith are provided. Such polymers may be derived from amino acids comprising suitable functional groups for associating the polymer to the nanoparticle. For example, in some embodiments, the polymer includes a polypeptide backbone (e.g., polyaspartic acid) with amino acid side groups (e.g., cysteine and/or methionine). Such a polymer can enable strong binding of the polymer to the nanoparticle surface via its multiple thiol groups, which can lead to excellent colloidal stability. Moreover, the carboxylic acid and amine functional groups of the polymer can facilitate attachment of binding partners (e.g., antibodies) to the polymer, which can allow the polymer-coated nanoparticle to be used in a variety of applications including protein detection and cell labeling.

### FIELD OF INVENTION

The present invention relates generally to polymers for coating nanoparticles, nanoparticles coated with polymers, and methods associated therewith.

### BACKGROUND

Colloidal nanocrystals have great importance in basic and applied research. Current research focuses on the synthesis, colloidal stability, biocompatibility and to conjugation chemistry of nanoparticles. Surfactant-mediated nucleation and growth can be important towards size control of nanoparticles in the range of 1-10 nm. Methods are available for the synthesis of near-monodisperse nanoparticles of quantum dots, noble metals, and metal oxides. For instance, the nanoparticles can be coated with a layer of surfactant molecules that protect them from further growth and external environment. However, these surfactants may also render the nanoparticles hydrophobic and/or prevent the nanoparticles from undergoing further chemical functionalization. Furthermore, the surfactant layer attached to the surface of the nanoparticles may be unstable to subsequent processing and conjugation chemistry. Accordingly, compositions and methods for synthesizing colloidally stable, water-soluble and robust nanoparticles with flexible surface chemistry is needed.

### SUMMARY OF THE INVENTION

Polymers for coating nanoparticles are provided, nanoparticles coated with polymers, and methods associated therewith are provided.

In one embodiment, a polymer is provided. The polymer comprises a polypeptide backbone functionalized with amino acid side groups that can bind to a surface of a nanoparticle, and that can participate in covalent attachment of a chemical or biological entity to the polymer, present in a sufficient quantity such that when the polymer is applied to a nanoparticle, at least a portion of the nanoparticle surface is coated with the polymer so as to form a single, isolated polymer-coated nanoparticle having a size of less than or equal to 10 nanometers, presenting for attachment functional groups able to participate in covalent attachment of a chemical or biological entity. In some cases, the polymer has a molecular weight of from about 10 kDa to about 20 kDa.

In another embodiment, a coated nanoparticle is provided. The coated nanoparticle comprises a nanoparticle comprising a colloidal or semiconductor material, and a polymer coating on at least a portion of a surface of the nanoparticle, the polymer coating comprising a polypeptide backbone functionalized with amino acid side groups.

In another embodiment, a method of forming a polymer-coated nanoparticle is provided. The method comprises selecting a nanoparticle and a polymer comprising a polypeptide backbone functionalized with amino acid side groups, the polymer comprising functional groups that can bind to a surface of the nanoparticle, and coating at least a portion of the nanoparticle surface with the polymer so as to form single, isolated polymer-coated nanoparticle having a size of less than or equal to 10 nanometers.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 shows a scheme for synthesizing a polymer having a polyaspartic acid backbone with cysteine side groups according to one embodiment of the invention;

FIG. 2 shows a schematic diagram of a nanoparticle coated with the polymer shown in FIG. 1 according to one embodiment of the invention;

FIGS. 3A-3D show absorption spectra of nanoparticles of different compositions and sizes having coatings of the polymer shown in FIG. 1 according to one embodiment of the invention;

FIG. 4 shows photographs of the polymer-coated nanoparticles used to obtain the spectra shown in FIG. 3 according to one embodiment of the invention;

FIGS. 5A-5D show representative TEM micrographs of various polymer-coated nanoparticles according to one embodiment of the invention;

FIG. 6 shows emission spectra of polymer-stabilized ZnS-capped CdSe quantum dots according to one embodiment of the invention;

FIG. 7 is a photograph of the quantum dots used to obtain the spectra of FIG. 6 according to one embodiment of the invention;

FIG. 8 shows a schematic diagram demonstrating h-IgG detection by polymer-stabilized quantum dot nanocrystals according to one embodiment of the invention;

FIG. 9 shows the results of h-IgG detection by polymer-stabilized quantum dot nanocrystals according to one embodiment of the invention; and

FIG. 10 shows labeling of 4T1 mouse breast cancer cells with polymer-coated quantum dot nanocrystals functionalized with anti-m-EGFR according to one embodiment of the invention. The inset shows a photograph obtained with control quantum dot nanocrystals that were not functionalized with anti-m-EGFR.

### DETAILED DESCRIPTION

Polymers for coating nanoparticles (e.g., colloid nanoparticles and quantum dots), nanoparticles coated with polymers, and methods associated therewith are provided. Polymers for coating nanoparticles, in the invention, are selected to have particular functional groups for immobilization to the nanoparticle, and for coupling an auxiliary species to the nanoparticle. It has been found that a particular molecular weight range for these polymers gives a surprising combination of superior particle coating capacity, and freedom from inter-particle agglomeration, and polymers within this molecular weight range are provided in one aspect of the invention.

Such polymers may be derived from amino acids comprising suitable functional groups for associating the polymer to the nanoparticle. For example, in some embodiments, the polymer includes a polypeptide backbone (e.g., polyaspartic acid) with amino acid side groups (e.g., cysteine and/or methionine). Such a polymer can enable strong binding of the polymer to the nanoparticle surface via its multiple thiol groups, which can lead to excellent colloidal stability. Moreover, selected side groups (e.g., carboxylic acid and amine functional groups) of the polymer can facilitate attachment of binding partners (e.g., antibodies) to the polymer, which can allow the polymer-coated nanoparticle to be used in a variety of applications including protein detection, cell labeling, and imaging.

One aspect of the invention includes a nanoparticle having a surface that is at least partially coated with a polymer. In some embodiments, the polymer forms a single monolayer on the nanoparticle surface. The inventors have discovered that in order to form single, isolated nanoparticles at least a portion of which is coated with a polymer, the polymer may have one or more of the following attributes (in one embodiment, the polymer has all of these attributes): a suitable molecular weight distribution; certain selected physical properties, such as charged groups and/or low hydrophobicity, to avoid aggregation of the polymer during preparation of the polymer-coated nanoparticle and of coated particles to each other after coating; and suitable functional groups that can bind to a surface of the nanoparticle and can serve as coupling points for attachment of selected auxiliary chemical or biological species (e.g., a binding partner). The functional groups for attachment of the polymer to the particle should be present in a sufficient quantity such that when the polymer is applied to the nanoparticle, at least a portion of the nanoparticle surface is coated with the polymer and the polymer resists detachment from the nanoparticle surface. In some embodiments, the same functional groups that facilitate attachment of the polymer to the nanoparticle also enable binding of a chemical or biological entity to the polymer. For instance, the polymer-coated-nanoparticle may participate in covalent attachment of an entity such as a binding partner to the polymer, which can be used to capture an analyte or the like which binds to the binding partner.

As described in more detail below, the molecular weight distribution of the polymer may be chosen such that it is high enough to form a coating on a nanoparticle but not so high as to cause agglomeration of the polymer. The polymer may have a molecular weight of, for example, from about 5-50 kilodaltons (kDa) or from about 10-20 kDa in other embodiments. In certain embodiments, polymers are chosen so as to form a single, isolated polymer-coated nanoparticle having a size of less than or equal to 10 nanometers. The molecular weight range of the polymer described in this aspect may be particularly suitable when nanoparticles with a particular size range are used (size being measured exclusive of the polymer coating). In such a case, nanoparticles of diameter ranging from, for example, about 1 nm to about 10 nm can be selected, and “diameter”, in this context, means diameter as measured by the technique of Scanning Electron Microscopy (SCM), Transmission Electron Microscopy (TEM) or particle size s analysis by Dynamic Light Scattering (DLS). “Diameter” in this context, in the case of non-spherical particles, means, for an individual particle, average of the several possible diameters of the particle.

In some embodiments, a suitable polymer for coating a nanoparticle comprises a polypeptide which may be optionally functionalized with various side groups. The to polymer may include a polypeptide backbone functionalized with amino acid side groups. In one particular embodiment, polyaspartic acid (also known as polyaspartate) and/or polyglutamic acid is reacted with a —NH-containing compound to form a polymer that can be used to coat a nanoparticle. The —NH-containing compound may include amino acids, i.e., molecules that contain both amine and carboxyl functional groups. Amino acids include alpha amino acids, molecules where the amino and carboxylate groups are attached to the same carbon (which is called the alpha-carbon). Advantageously, amino acids are water soluble and include functional groups that can allow binding of the polymer to a nanoparticle and/or allow further functionalization of polymer. Examples of amino acids are described in more detail below.

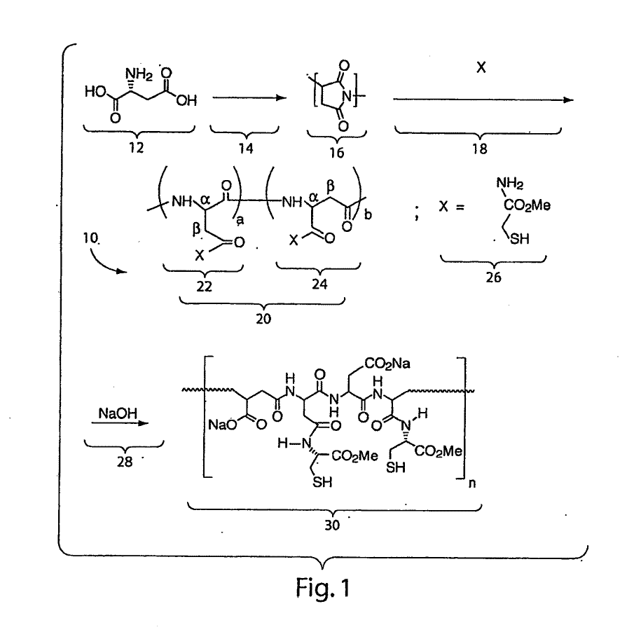

FIG. 1 shows a scheme for synthesizing a polymer comprising a polyaspartic acid backbone with various side groups according to one embodiment of the invention. (Such a method can also be used for synthesizing a polymer comprising a polyglutamic acid backbone with various side groups in other embodiments.) As illustrated in scheme 10, aspartic acid 12 (e.g., L-, D-, or DL-aspartic acid) may be reacted under suitable conditions 14 to form polysuccinimide 16. Methods of forming polysuccinimide from aspartic acid are known by those of ordinary skill in the art. For example, aspartic acid may be heated at a temperature greater than 180° Celsius in the presence of an acid (e.g., phosphoric acid) to produce polysuccinimide via a polycondensation reaction. In other cases, lower temperatures and shorter reaction times are possible by using catalysts. Next, polysuccinimide 16 may be reacted with a suitable side group X under conditions 18 (optionally, in the presence of a catalyst) to cause ring-opening of the polysuccinimide and its reaction with side group X. This reaction can result in the synthesis of a modified polyaspartic acid polymer 20. Because the ring opening of polysuccinimide to polyaspartic acid can occur in two possible ways, polymer 20 may include two polymer linkages: an alpha-linkage 22 and/or a beta-linkage 24. A polymer described herein for coating a nanoparticle may have any suitable proportions or combinations of alpha and beta linkages. In one particular embodiment, polysuccinimide 16 is reacted with a protected amino acid such as methylcysteine 26 under basic conditions 28 to form polymer 30 (cysteine-functionalized polyaspartic acid), which comprises a polyaspartic acid backbone with methylcysteine side groups. In other embodiments, side groups X of polymer 20 may include other NH-containing compounds such as other amino acids (e.g., methionine).

As mentioned above, in some embodiments, the formation of a polypeptide with suitable side groups may take place in the presence of a catalyst. In such embodiments, the amount of catalysts present in the reaction mixture can affect the molecular weight of the resulting polymer. For instance, in certain embodiments, a relatively high amount of catalyst can result in a polymer having a lower molecular weight while lower amounts of catalysts can cause the polymer to have a higher molecular weight (for reasons that will be apparent to those of ordinary skill in the art). The presence of the catalyst may also cause the formation of a polymer having a substantially narrow molecular weight distribution (e.g., within less than or equal to 10 kDa, within less than or equal to 5 kDa, or within less than or equal to 3 kDa). Examples of suitable catalyst include phosphoric acid, polyphosphoric acid, sulfuric acid, sulfonic acids (para toluene sulfonic acid), Lewis acids (e.g., Scandium triflate) Bronsted acids, and biocatalysts such as enzymes.

Amino acids that can be used to form a polymer (e.g., either a backbone and/or a side group of a polymer) can be natural or synthetic. Examples of suitable natural amino acids include glycine, alanine, valine, leucine, isoleucine, methionine, proline, phenylalanine, tryptophan, serine, threonine, asparagine, glutamine, tyrosine, cysteine, lysine, arginine, histidine, aspartic acid, glutamic acid. In some cases, amino acids and/or their derivatives with the following classifications can be used to form a polymer-coated nanoparticle: amino dicarboxylic acids (e.g., aspartic acid, glutamic acid and cystine (an oxidized dimeric form of cysteine)), neutral amino acids (e.g., glycine, alanine, beta-alanine, valine, leucine, isoleucine, methionine, cysteine, aminocaproic acid (a derivative of lysine), asparagine, isoasparagine, glutamine and isoglutamine), N-methylamino acids (e.g., N-methylglycine and N-methylcystein), amino sulfonic acids (e.g., taurine, a derivative of cysteine)), hydroxy carboxylic (e.g., hydroxyproline, serine and threonine), imino carboxylic acids (e.g., proline and iminodiacetic acid), aromatic and heterocyclic amino acids (e.g., anthranilic acid, tryptophan, tyrosine and histidine), amino tricarboxylic acids (e.g., alpha-beta-aminotricarballylic acid), and/or basic diamino carboxylic acids (e.g., lysine, lysine hydrochloride, arginine, histidine and alpha-aminocaprolactam). Amino acids may be protected or non-protected.

In some embodiments, amino acids used to form either the backbone and/or side group of a polymer is chosen based upon its charge, hydrophobicity and/or polarity, in part to prevent polymer and/or inter-nanoparticle agglomeration. Examples of suitable non-polar and hydrophobic amino acids include phenylalanine, methionine, tryptophan, isoleucine, valine, leucine, alanine, and proline. Examples of suitable negatively charged (polar and hydrophilic) amino acids include aspartic acid and glutamic acid. Examples of suitable amino acids that are polar and hydrophilic but uncharged include cysteine, asparagine, glutamine, threonine, tyrosine, serine, and glycine. Examples of suitable positively charged (polar and hydrophilic) amino acids include histidine, lysine, and arginine. In certain embodiments of the invention, a polymer used for coating a nanoparticle includes a backbone formed of a negatively charged amino acid. Side groups of the polar may include, in some cases, a polar (hydrophilic) amino acid that may be charged or uncharged.

In other embodiments, a polymer backbone comprising a polypeptide (e.g., polyaspartic acid) is reacted with a —NH-containing compound that is not an amino acid to form a polymer that can be used to coat a nanoparticle. Non-limiting examples of such compounds include glucoseamine, chitosan, PEGylated amines (polyethylene glycol having amine groups), nucleophilic aliphatic, aromatic and heterocyclic amines, oxygen nucleophiles and carbon nucleophiles, aliphatic, aromatic and heterocyclic diamines, and aminoalcohols. In yet other embodiments, one or more of such compounds can form all or at least a portion of the polymer backbone:

The monomers used to form the backbone and/or side groups of the polymer may be chosen based on the presence of one or more functional groups that may allow the nanoparticle to have a desired property such as, for example, water solubility, reactivity, biocompatibility, and/or availability for bio-conjugation and/or modification. In some instances, the side groups may be chosen at least in part by the material composition of the nanoparticle to which the polymer coating is formed.

Affinity between functional groups and materials used to form nanoparticles can be determined by simple screening tests as described in more detail below. In certain embodiments, affinity between a particular chosen functional group and a surface of the nanoparticle may be relatively weak, however, a large number of such associations can cause adequate coating of the nanoparticle with the polymer. In other cases, stronger functional group interactions can allow a lower number of surface-attaching functional groups to be used. In some embodiments, the side groups are selected not only to include functional groups that attach the polymer coating to the nanoparticle surface, but also to allow covalent attachment of a chemical or biological entity to the coating. Functional groups that allow attachment of the polymer coating to a nanoparticle surface and those that allow covalent attachment of an entity to the polymer may be the same in some embodiments, or different in other embodiments. The composition of the polymer may also be selected such that when it forms a coating on a nanoparticle, the coating resists separation from the nanoparticle under conditions of covalent attachment of the chemical or biological entity to the coating. In other embodiments, the backbone and/or side groups are chosen so as to cause poor polymer-polymer interaction. For example, the polymer may be chosen to have low hydrophobicity to avoid hydrophobic interactions with one another during formation of the polymer-coated nanoparticles. The backbone and/or side groups may also be charged (positively or negatively) to avoid aggregation of the polymer. In some instance, the backbone and/or side groups are polar but uncharged. Advantageously, polymers that are polar and/or charged (e.g., have low hydrophobicity) can form single monolayer coatings on nanoparticles in certain embodiments. Sometimes, all or a combination of the factors listed above are considered for choosing an appropriate polymer or polymer precursors.

As mentioned above, side group of polymers of the invention may be chosen based on suitable functional groups that can allow attachment of the polymer to the surface of the nanoparticle, and allow attachment of one or more auxiliary chemical or biological species (e.g., a binding partner) to the polymer. Some functional groups facilitate immobilization to a nanoparticle (and the nanoparticle and functional group should in this case be selected together for this purpose, for example according to a screening test described herein), and some facilitate attachment to the auxiliary entity. In some cases, a single type of functional group serves both purposes. Most functional groups that facilitate immobilization of the polymer to the nanoparticle can also serve as immobilization points for auxiliary entities, but some functional groups that can serve as attachment points for auxiliary entities do not serve well as points for attachment to the nanoparticle surface (and, in more limited cases, the opposite is true). What roles each functional group serves is to be considered in selecting a frequency/density of presence of each (if more than one) functional group type on the polymer (e.g., amount of functional group present per polymer repeat unit). If the same functional group serves both roles, then its repeat frequency on the polymer backbone typically will be relatively higher, and its frequency should be chosen such that, after coating of the nanoparticle with the polymer, sufficient free functional group remains (not consumed in the role of attachment of the polymer to the nanoparticle) so as to provide a desired concentration of point of attachment for an auxiliary entity on the polymer coated nanoparticle. If, for example, a particular functional group serves only as a point for covalent attachment of an auxiliary entity, then its frequency typically will be relatively lower, and can be selected based only on the concentration of auxiliary entity attachment point desired on the polymer coated nanoparticle. Generally, functional groups should be present in sufficient quality and quantity such that when the polymer is applied to a nanoparticle, at least a portion of the nanoparticle surface is coated with the polymer so as to form a single, isolated polymer-coated nanoparticle (e.g., having a size of less than or equal to 10 nanometers). The density of functional groups can be based on a number of suitable binding sites relative to the number of monomers used to form the backbone of the polymer (selected, for one or a number of different functional groups, individually or together, according to principles discussed immediately above and elsewhere herein). For instance, the ratio of binding sites to the number of monomers used to form the backbone may be greater than or equal to 0.1:1, greater than or equal to 0.3:1, greater than or equal to 0.5:1, greater than or equal 1:1, or greater than or equal to 2:1. In other embodiments, the density of binding sites can be determined based on the ratio of molecular weight of a repeat unit to the molecular weight of the binding site. Such ratios may be between, for example, 1:1-3:1, 4:1-7:1, 8:10-10:1, or 11:1-15:1. For example, the molecular weight of an aspartic acid and cystine repeat unit is approximately 250 g/mol and the molecular weight of a sulfur atom, which may be used to bind the polymer to a nanoparticle surface, is 32 g/mol. The ratio of molecular weight of the repeat unit to that of the binding site is, therefore, approximately 8:1. In other embodiments, other binding sites such as carboxylate and amine groups can be used to attach the polymer to a nanoparticle surface and the densities of these binding sites may vary as the backbone and/or side groups of the polymer may include one or more such functional groups. Selection of functional groups (with a single type serving both roles, or different groups serving different roles of attachment to nanoparticle and to auxiliary entity), and functional group frequency or ratio to backbone repeat unit, can be selected by those of ordinary skill in the art based, at least in part, on descriptions and screening tests described herein.

Although the primary description herein involves polymers having backbones formed of a single monomer (e.g., a polypeptide formed of a single amino acid), it should be understood that other forms of polymers that can be used for coating nanoparticles are also possible. For instance, in some instances, the backbone of the polymer is a copolymer, a polymer that includes two distinct monomers. In some embodiments, the copolymer includes a polypeptide copolymerized with a monomer that is not an amino acid. For example, the polymer may be poly(lactic acid-co-aspartic acid). Such copolymers can be functionalized with various side groups as described herein. In other embodiments, terpolymers, polymers that include three distinct monomers (e.g., poly(L-lactic acid)/poly(ethylene oxide)/poly(L-aspartic acid)) may be used to coat nanoparticles. The terpolymers may also be functionalized with various side groups. Such polymers may be in the form of for example, diblock copolymers and multiblock polymers. In yet other embodiments, polypeptides including two or more of the natural amino acids (and/or derivatives thereof) are used to form a polymer backbone (e.g., polyaspartic acid-co-polyglutamic acid).

Polymers described herein may have any suitable molecular weight. In some embodiments, the molecular weight of a polymer is chosen such that when the polymer is combined with a nanoparticle, at least a portion of the nanoparticle surface is coated with the polymer so as to form a single, isolated polymer-coated nanoparticle. Such a nanoparticle may have a size of, for example, less than or equal to 100 nanometers, less than or equal to 50 nanometers, less than or equal to 25 nanometers, or less than or equal to 10 nanometers. In some embodiments, the molecular weight of the polymer is high enough to coat the nanoparticle (e.g., to form a monolayer of the polymer on the nanoparticle surface), but low enough as to not cause polymer agglomeration). Such a molecular weight range may be, for example, between 5-50 kDa, between 10-30 kDa, between 10-20 kDa, between 10-15 kDa, or between 5-20 kDa. Of course, a suitable molecular weight range may depend upon factors such as the size of the nanoparticle, the composition of the polymer, the desired thickness of the polymer coating, and the method of attachment of the polymer to the nanoparticle surface.

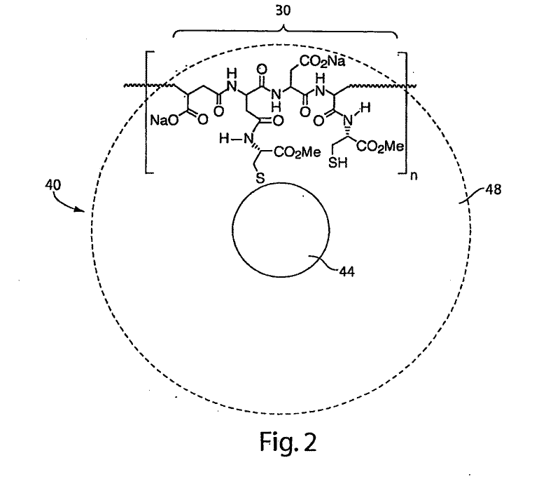

In some embodiments, polymers described herein such as those shown in FIG. 1 can be used to form polymer-coated nanoparticles. For instance, in the embodiment illustrated in FIG. 2, single, isolated polymer-coated nanoparticle 40 includes a nanoparticle 44 and a coating 48 formed of polymer 30, which has a polyaspartic acid backbone with cysteine side groups. Of course, nanoparticle 44 may be coated with other polymers such as other amino acid-functionalized polypeptides or other polymers described herein. In certain embodiments, nanoparticle 44 comprises a colloidal material or a semiconductor material. I.e., nanoparticle 44 may be a colloidal nanoparticle (e.g., a gold (Au) or silver (Ag) nanoparticle) or a quantum dot (i.e., a semiconductor nanocrystal). Polymer 30, which may include thiol (—SH) groups can, in such embodiments, attach to a surface of the nanoparticle via a sulfur-metal and/or a sulfur-semiconductor bond. As described in more detail below, other forms of attachment between polymer coating 48 and nanoparticle 44 may be used depending on, for example, the material composition of coating 48 and/or nanoparticle 44, the available number of binding sites of coating 48, the method of synthesis of nanoparticle 44, the method of coating nanoparticle 44, and the particular application and/or desired properties of the polymer-coated nanoparticle.

Although FIG. 2 shows coating 48 completely coating core 44, in other embodiments, coating 48 may coat only portions of nanoparticle 44. Furthermore, although a single nanoparticle 44 is shown, in some cases, a nanostructure can include several nanoparticles coated by coating 48. In further embodiments, nanoparticle 44 can have multiple coatings, e.g., of two or more different polymers, of non-polymeric materials such as silica, and/or combinations of polymers and non-polymers. It should be understood that FIG. 2 is a schematic diagram and that the compositions and dimensions of the polymer-coated nanoparticles described herein can vary.

A variety of methods may be used to form nanoparticle 40. In one embodiment, polymer-coated nanoparticles are prepared by a ligand-exchange process. In another embodiment, polymer-coated nanoparticles are prepared by the direct reduction of metal salt in the presence of the polymer. Such methods are described in more detail below. Other methods for coating nanoparticles with polymers described herein are also possible.

In one particular embodiment, nanoparticle 44, which may be, for example, a gold or silver nanoparticle or a quantum dot nanocrystal, is coated with a polymer by a ligand-exchange method. In such a procedure, the nanoparticle can be made using methods known to those of ordinary skill in the art. Optionally, the nanoparticle may be synthesized to include a passivation layer. A “passivation” layer is a material associated with the surface of a nanoparticle that serves to eliminate energy levels at the surface that may act as traps for electrons and holes that degrade the luminescent properties of the nanoparticle. A passivation layer may include a layer of surfactant. For instance, gold and/or silver nanoparticles can be prepared to include long-chain fatty acid/fatty amine surfactants as ligands. Quantum dot nanocrystals may be prepared using known methods to include ligands such as fatty amines, trioctylphosphine oxide (TOPO) and trioctylphosphine (TOP). Of course, other compounds that may be used as passivation layers may also be used to coat nanoparticle 44.

In some embodiments, the passivation layer may be formed of a material that is non-conductive and/or non-semiconductive. For example, the passivation layer may be of a material that does not exhibit a higher band gap than a nanoparticle which it surrounds. In specific embodiments, the passivation layer may be non-ionic and non-metallic. A non-conductive material is a material that does not transport electrons when an electric potential is applied across the material. The material forming the passivation may be hydrophilic or hydrophobic depending on the desired properties of the nanoparticle.

In certain embodiments, the passivation layer can be comprised of, or consist essentially of, a compound exhibiting a nitrogen-containing functional group, such as an amine. The amine may be bound directly or indirectly to one or more silicon atoms such as those present in a silane or other silicon polymer. The silanes may include any additional functional group such as, for example, alkyl groups, hydroxyl groups, sulfur-containing groups, or nitrogen-containing groups. Compounds comprising the passivation layer may be of any size but typically have a molecular weight of less than about 500 or less than about 300. Examples include amino silanes such as amino propyl trimethoxysilane (APS).

After the nanoparticle has been prepared with a ligand to form a passivation layer, the nanoparticles, which may be rendered hydrophilic, can then be dissolved in an aqueous in nonaqueous (reverse) microemulsion, using for example, an ionic or non-ionic surfactant. Non-ionic surfactants include, for example, polyphenyl ethers, such as IGEPAL CO-520, while ionic surfactants include, for example, dioctyl sulfosuccinate sodium salt (AOT). After introduction of the passivated nanoparticle into the reverse emulsion, the ligand can be partially or completely exchanged for the ionic or non-ionic surfactant (e.g., due, in part, to a higher concentration of the ionic or non-ionic surfactant in the reverse emulsion) such that the nanoparticle is at least partially coated with the ionic or non-ionic surfactant. Next, concentrated aqueous polymer solution (e.g., polymer 20 or 30 of FIG. 1) can be introduced for ligand exchange with the ionic or non-ionic surfactant to form a polymer-coated nanoparticle.

It should be understood that in some embodiments, nanoparticles that do not include a passivation layer can be used as precursors to polymer-coated nanoparticles.

In one particular embodiment, a nanoparticle prepared with a trioctylphosphine oxide (TOPO) surfactant as a passivation layer is combined with IGEPAL in an aqueous in non-aqueous reverse microemulsion. TOPO includes a hydrophilic end comprising phosphine oxide while IGEPAL includes a hydrophilic end comprising polyoxyethylene (PEO). After introduction of the TOPO nanoparticle into the reverse emulsion, the TOPO can be partially or completely exchanged for IGEPAL. Concentrated aqueous polymer solution such as a solution containing cysteine- and/or methionine-functionalized polyaspartic acid can then be introduced for ligand exchange with the IGEPAL to form polymer-coated nanoparticle 40 of FIG. 2.

Surfactants other than IGEPAL may be used and may be varied, in part, depending upon the nanoparticle material, whether the nanoparticle is capped and with what ligand, and the method of forming the coated nanoparticle (e.g., a regular emulsion compared to a reverse emulsion). For the preparation of water soluble (hydrophilic) polymer-coated nanoparticles, preferred surfactants include those that can be exchanged for TOPO or other surfactants that are used to cap the nanoparticle and that also provide enough hydrophilicity to draw the core into aqueous portions of the micro-emulsion, thus providing an environment for the formation of the polymer coating.

In certain embodiments including the use of quantum dots as the nanoparticle, a small amount of aqueous tetramethyl ammonium hydroxide solution or other suitable compound can be added to facilitate the ligand exchange. For instance, in some embodiments, the polymer is exchanged within minutes, e.g., as observed by the color change of the Au and Ag systems. After mixing (e.g., 5 min of vortex), ethanol or another suitable solvent can be added to disrupt the reverse microemulsion. The precipitated particle can be separated by centrifuging, followed by repeated washing (e.g., with cyclohexane and ethanol sequentially). The resulting nanoparticles can be dissolved in water or buffer solutions. The buffer solution of the polymer-stabilized nanoparticles may be stable for long periods of time. For instance, in some embodiments, polymer-stabilized nanoparticles are stable in an aqueous solvent for to greater than 1 day, greater than 1 week, greater than 1 month, greater than 3 months, greater than 6 months, or greater than 1 year.

In some instances, colloidal (e.g., Au and Ag) nanoparticles can be synthesized by direct reduction of the respective metal salts in the presence of a polymer. In such methods, aqueous solutions of metal salts and polymers can be mixed by stirring, followed by the injection of a reducing agent such as a sodium borohydride solution.

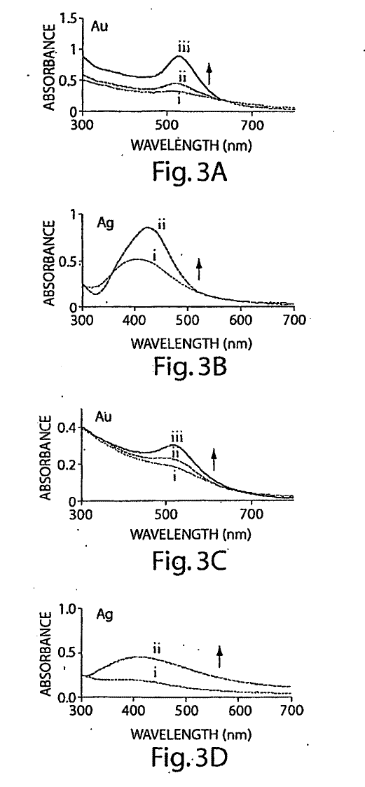

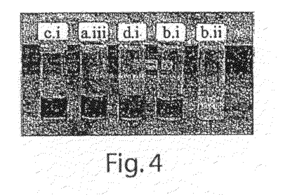

FIGS. 3A-3D show absorption spectra of solutions containing gold (FIGS. 3A, 3C) and silver (FIGS. 3B, 3D) nanoparticles coated with a polymer comprising a polyaspartic acid backbone having cysteine functional groups (e.g., polymer 30 of FIG. 1): The nanoparticles were prepared by the ligand-exchange method (FIGS. 3A, 3B) and by direct synthesis in the presence of polymer (FIGS. 3C, 3D) using final polymer concentrations of (i) 1.0%, (ii) 0.1% and (iii) 0.05%. The size of the nanoparticles were varied by changing the polymer concentration. The arrows in the figures indicate increasing size of the polymer-coated nanoparticle as the polymer concentration decreased. FIG. 4 shows photographs of the polymer-coated nanoparticles used to obtain the spectra shown in FIG. 3.

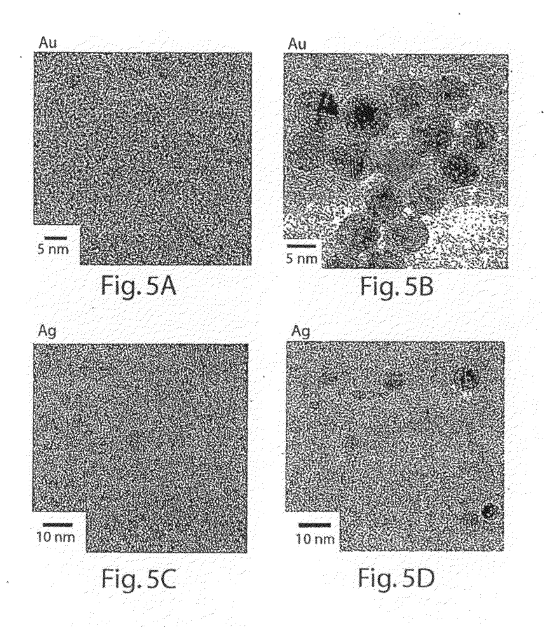

FIGS. 5A-5D show transmission electron microscopy (TEM) micrographs of Au (FIGS. 5A, 5B) and Ag (FIGS. 5C, 5D) cysteine-functionalized polyaspartic acid-coated nanoparticles of different sizes prepared by the ligand-exchange method (FIGS. 5A-5C) and by direct synthesis in the presence of the polymer (FIG. 5D). As illustrated in these figures, the polymer-coated nanoparticles can be fabricated to have average sizes (e.g., diameters) of less than or equal to 10 nanometers in some embodiments, and less than or equal to 5 nanometers in other embodiments. For instance, the average sizes of the polymer-coated nanoparticles in FIG. 5 were measured to be 2-3 nm (FIG. 5A), 5 nm (FIG. 5B), 3-4 nm (FIG. 5C), and 5-6 nm (FIG. 5D). In each of these figures, the thickness of polymer coating was 1-2 nm. Also shown in FIGS. 5A-5D, the polymer-coated nanoparticles described herein may be substantially monodispersed.

In certain embodiments of the invention, a thin coating of a polymer (e.g., a water-soluble polymer) on a nanoparticle can be prepared. For instance, the coating may have a thickness of less than or equal to 10 nm, less than or equal to 5 nm, less than or equal to 3 nm, less than or equal to 2 nm, less than or equal to 1 nm, less than or equal to 0.5 nm, or less than or equal to 0.3 nm. Thin coatings are particularly suitable for applications that require very small nanoparticles (e.g., less than about 6 nm) such as labeling of small structures. Small nanoparticle structures may also be useful for applications involving fluorescence resonance energy transfer (FRET). In such cases, nanoparticles having water-soluble coatings can be used in FRET applications to study, for example, protein-protein interactions, protein-DNA interactions, and protein conformational changes.

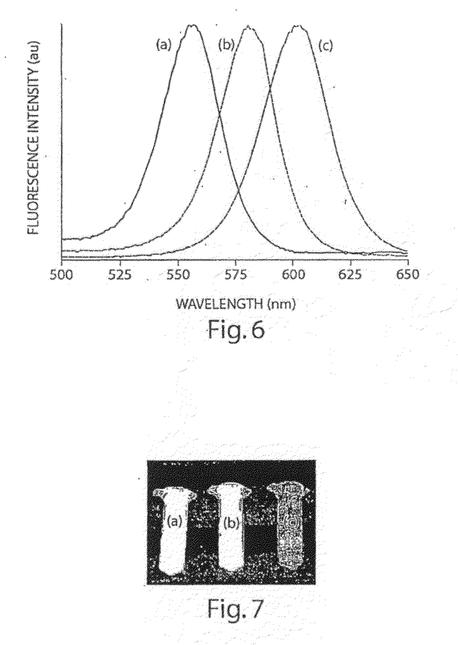

FIG. 6 shows emission spectra of cysteine-functionalized polyaspartic acid-coated ZnS-capped CdSe quantum dots and FIG. 7 is a photograph of the quantum dots. As shown in these illustrative embodiments, the polymer-coated nanocrystals may have fluorescence emissions that are tunable between 500 nm and 650 nm by, for example, varying the size of the nanoparticles. As is known in the art, other ranges of emissions are possible by choosing nanoparticles with different material compositions.

As shown in FIG. 6 and as described in more detail below, the polymer-coated nanoparticles can emit electromagnetic radiation having narrow bandwidths. For instance, the bandwidths may be less than 50 nanometers, less than 40 nanometers, or less than 30 nanometers. Furthermore, the polymer-coated nanoparticles may have quantum yields (QY) of greater than or equal to 10%, greater than or equal to 15%, greater than or equal to 20%, greater than or equal to 25%, greater than or equal to 30%, or greater than or equal to 35% in aqueous solution. As described in more detail below, such nanoparticles may have a variety of applications such as, for example, fluorescent labels for biological imaging applications (e.g., as fluorescent tags for biological and/or chemical materials).

As described above, in certain embodiments, nanoparticles described herein can include a coating of a polymer on at least a portion of the nanoparticle surface. In some cases, the polymer (and, therefore, the nanoparticle to which the polymer is coated) is water-soluble; that is, the polymer may include one or more functional groups that render the polymer/nanoparticle water-soluble. The term “water soluble”, in this context, is used herein as it is commonly used in the art to refer to the dispersion of a nanoparticle in an aqueous or water-soluble environment. “Water soluble” does not mean, for instance, that each material is dispersed at a molecular level. A nanoparticle can be composed of several different materials and still be “water soluble” as an integral particle.

Suitable water-soluble polymers may comprise functional groups such as carboxyl, amine, amide, imine, aldehyde, hydoxyl groups, the like, and combinations to thereof. Such functional groups may define terminating groups of a coating (or at least partial coating) of a nanoparticle described herein. For instance, a coating may be assembled, or may self-assemble, in association with a surface of a nanoparticle such that a particular functional group is primarily or exclusively presented outwardly relative to the nanoparticle, and an entity interacting with the nanoparticle in a standard chemical or biochemical interaction first or primarily encounters that functional group. For example, in one embodiment, an carboxylate-terminating coating on a nanoparticle will primarily or exclusively present, to a species in a standard chemical or biochemical interaction with the nanoparticle, a carboxylate functionality.

In some particular embodiments, biocompatible water-soluble polymers are particularly suitable for coating nanoparticles that are used for interaction with cells (e.g., mammalian or bacterial cells) and/or biological material including nucleic acids, polypeptides, etc. For instance, nanoparticles coated with amino acid-based polymers may be more biocompatible and less cytotoxic than other water-soluble nanoparticles. In some cases, water-soluble polymers that can be incorporated into an aqueous synthesis of nanoparticles can produce water-soluble nanoparticles that are more biocompatible and/or less cytotoxic than nanoparticles prepared through organic or organometallic synthesis routes.

The polymer may interact with the nanoparticles to form a bond with the nanoparticle, such as a covalent bond, an ionic bond, a hydrogen bond, a dative bond, a coordination bond, or the like. The interaction may also comprise Van der Waals interactions. Sometimes, the polymer interacts with the nanoparticle by chemical or physical adsorption (i.e., chemisorption and physisorption, respectively). If desired, nanoparticles may be coated with one or more molecules (e.g., a surfactant) prior to being coated with a polymer in order to facilitate attachment of the polymer to the nanoparticle surface.

In some embodiments, the polymer may be crosslinked to impart stability of the polymer on the nanoparticle surface. Various methods of crosslinking can be used (e.g., by exposure to UV radiation, heat, and crosslinking agents) and determined by those of ordinary skill in the art.

In some embodiments, the polymer coating may be appropriately functionalized to impart desired characteristics (e.g., surface properties) to the nanoparticle. For example, the coating may be functionalized/derivatized to include compounds, functional to groups, atoms, or materials that can alter or improve properties of the nanoparticle. In other embodiments, the coating may comprise functional groups which can specifically interact with an analyte to form a covalent bond. The coating may include compounds, atoms, or materials that can alter or improve properties such as compatibility with a suspension medium (e.g., water solubility, water stability, i.e., at certain pH ranges), photo-stability, and/or biocompatibility.

Accordingly, in certain embodiments, nanoparticles such as colloidal nanoparticles and/or quantum dots are coated with a polymer including multiple thiol and carboxyl groups (e.g., cysteine- and/or methionine-functionalized polyaspartic acid). Such functional groups can facilitate excellent colloidal stability and solubility of the nanoparticles in aqueous solution. A stronger interaction of thiols may be expected to occur with Au and Ag nanoparticles than with quantum dots due to the favorable interaction between Au—S and Ag—S than with certain materials used to form the quantum dots. However, in some embodiments, thiols can attach to the surface of quantum dots of certain compositions, such as quantum dots including zinc (e.g., ZnS capped CdSe quantum dots). In these embodiments, sulfur can interact favorably with the Zn ions of the quantum dots; this interaction may be dependent on the solution pH. For instance, in some embodiments, a pH of greater than 6 (e.g., 7-10) can allow favorable interaction between thiol and zinc atoms. In some instances, one would expect that thiols would leach from the quantum dot surface in the presence of competitive ions and salts, and the nanocrystals would be precipitated with a rapid quenching of fluorescence. However, earlier works have showed that in some cases, an increased number of thiol groups in a molecule can provide greater stability. Accordingly, polymers including multiple thiol groups (e.g., in either the backbone and/or side groups of the polymer) may improve the effectiveness of ligand capping, and can facilitate binding of the polymer to certain nanoparticle surfaces. Such a coating can also restrict further growth and/or agglomeration of the polymer-coated nanoparticle during synthesis (e.g., after a ligand-exchange process).

It should be understood that thiol groups may interact favorably with other elements used to form nanoparticles such as magnetic nanoparticles and quantum dots, and, as a result, polymers described herein may be used to coat various nanoparticles having different material compositions. In other embodiments, polymers described herein can attach to nanoparticles by other functional groups such as, for example, to carboxylate, alcohol, amine, and silane groups which may be charged or uncharged.

Those of ordinary skill in the art can determine favorable interactions between functional groups that can be used to attach the polymer to a nanoparticle surface. For instance, bond energies between elements are known and can be used to determine the likelihood of attachment of the polymer to the nanoparticle. For example, the gold-sulfur bond has a bond energy of about 30-40 kcal/mol, which can cause relatively strong attachment between a polymer including a thiol and to a gold nanoparticle. However, as attachment may depend upon factors such as salt concentration and temperature, one may choose to perform a screening test to determine particular conditions for coating the nanoparticles. Simple screening tests such as the following can be performed. In one embodiment, a polymer that may be used to form a coated nanoparticle may be positioned on a surface (e.g., a bulk surface) of a material used to form the nanoparticle. The adhesiveness of the polymer layer or force required to remove the polymer layer from a unit area of a surface can be measured (e.g., in N/m²) using a tensile testing apparatus or another suitable apparatus. Surface plasmon resonance (SPR), X-ray photoelectron spectroscopy (XPS), and other surface techniques can also be performed to determine a characteristic of the surface (e.g., the thickness of a polymer layer on the surface) and/or presence or absence of a polymer layer on the surface. Such experiments may be performed in the presence of conditions used for attaching the polymer to the nanoparticle (e.g., in the presence of buffers, salts, certain temperatures) to determine the influence of the conditions on adhesion. The experiments can also be performed in the presence of other molecules/entities that may compete with the polymer for the material surface. In other embodiments, simple screening tests can include choosing particular polymers and nanoparticles having various material compositions, combining the materials using a known method for attaching the polymer to the nanoparticle surface (e.g., the ligand exchange method or by direct reduction of metal salts in the presence of the polymer), optionally varying a condition such as pH, temperature, concentration of reactant, and duration of the reaction. The nanoparticles can then be imaged using techniques such as transition electrons microscopy to determine whether the polymer adequately adhered to the nanoparticle. Other simple tests are known and can be conducted by those of ordinary skill in the art.

In some embodiments, a polymer-coated nanoparticle can interact with an analyte. The term “analyte,” may refer to any chemical, biochemical, or biological entity 10. (e.g., a molecule) to be analyzed. In some instances, the analyte is a chemical or biological analyte. In certain embodiments, polymer-coated nanoparticles described herein have high specificity for an analyte, and may be, for example, a chemical and/or biological sensor, or a small organic bioactive agent (e.g., a drug, agent of war, herbicide, pesticide, etc.).

A polymer-coated nanoparticle may associate with an analyte to form a bond with the analyte, such as a covalent bond (e.g., carbon-carbon, carbon-oxygen, oxygen-silicon, sulfur-sulfur, phosphorus-nitrogen, carbon-nitrogen, metal-oxygen or other covalent bonds), an ionic bond, a hydrogen bond (e.g., between hydroxyl, amine, carboxyl, thiol and/or similar functional groups, for example), a dative bond (e.g., complexation or chelation between metal ions and monodentate or multidentate ligands), a coordination bond (e.g., metal-sulfur), or the like. The association may also comprise Van der Waals interactions. In one embodiment, the association comprises forming a covalent bond with an analyte.

The coating may also associate with an analyte via a binding event between pairs of biological molecules/entities (i.e., binding partners). For example, the coating may comprise an entity, such as biotin that specifically binds to a complementary entity, such as avidin or streptavidin, on a target analyte. The entity may be attached to the coating by any suitable means such as the ones described above (e.g., via a covalent bond, ionic bond, hydrogen bond, dative bond, van der Waals interactions, and/or combinations thereof). In some embodiments, the entity is attached to the polymer coating directly (e.g., a functional group of the polymer may form a bond with a functional group of the entity). In other embodiments, the entity is attached to the polymer indirectly, such as via a coupling reagent or linker molecule. Common coupling reagents and linker molecules can be used and are known by those of ordinary skill in the art.

A polymer-coated nanoparticle may comprise one or more suitable functional groups and/or entities that acts as a binding site for an analyte. In some embodiments, the binding site comprises a biological or a chemical molecule/entity able to bind to another biological or chemical molecule/entity in a medium, e.g., in solution. For example, the binding site may be capable of biologically binding an analyte via an interaction that occurs between pairs of biological molecules/entities including proteins, nucleic acids, glycoproteins, carbohydrates, hormones, and the like. Specific examples include an antibody/peptide pair, an antibody/antigen pair, an antibody fragment/antigen pair, an antibody/antigen fragment pair, an antibody fragment/antigen fragment pair, an antibody/hapten pair, an enzyme/substrate pair, an enzyme/inhibitor pair, an enzyme/cofactor pair, a protein/substrate pair, a nucleic acid/nucleic acid pair, a protein/nucleic acid pair, a peptide/peptide pair, a protein/protein pair, a small molecule/protein pair, a glutathione/GST pair, an anti-GFP/GFP fusion protein pair, a Myc/Max pair, a maltose/maltose binding protein pair, a carbohydrate/protein pair, a carbohydrate derivative/protein pair, a metal binding tag/metal/chelate, a peptide tag/metal ion-metal chelate pair, a peptide/NTA pair, a lectin/carbohydrate pair, a receptor/hormone pair, a receptor/effector pair, a complementary nucleic acid/nucleic acid pair, a ligand/cell surface receptor pair, a virus/ligand pair, a Protein A/antibody pair, a Protein G/antibody pair, a Protein L/antibody pair, an Fc receptor/antibody pair, a biotin/avidin pair, a biotin/streptavidin pair, a drug/target pair, a zinc finger/nucleic acid pair, a small molecule/peptide pair, a small molecule/protein pair, a small molecule/target pair, a carbohydrate/protein pair such as maltose/MBP (maltose binding protein), a small molecule/target pair, or a metal ion/chelating agent pair. In some cases, the nanoparticles may be used in applications such as drug discovery, the isolation or purification of certain compounds, and/or implemented in assays or high-throughput screening techniques.

One of the key challenges in nanoparticle applications lies in the colloidal stability of the nanoparticles during the attachment of a binding site (e.g., one of a binding partner pair) to the nanoparticle (which can subsequently be used for the detection of the complementary binding partner pair). For instance, generally, bi-functional thiol molecules that can be functionalized with a binding site can produce water-soluble nanoparticles, which may be stable in buffer solutions or under high salt concentrations; however, the nanoparticles may aggregate or grow during functionalization/conjugation. This aggregation or growth may occur due to leaching of the surface chemisorbed thiol groups in the presence of competitive ligands from the conjugating proteins or other molecules that act as binding sites. In other instances, the reagents involved in conjugation chemistry can react with the capping ligands and even with the nanoparticles themselves. Furthermore, the ligand protection may not be sufficient for drastic conditions associated with conjugation chemistry (e.g., purification and processing steps such as centrifuge, dialysis, size exclusion chromatography, use of organic solvent, acidic or basic pH, etc.). However, as described herein, a polymer may be chosen to have certain side groups (and functional groups) such that when the polymer is used to coat a nanoparticle, the coating resists separation from the nanoparticle under conditions of covalent attachment of the chemical or biological entity to the coating.

As described above, simple screening tests can be performed to determine whether a polymer detaches from a surface under certain conditions. For example, a particular polymer may be used to coat a bulk surface (formed of a material used to form the nanoparticle), and may be put under certain conditions such as those associated with conjugation chemistry. The polymer surface can be compared before and after being treated with such conditions to determine whether the polymer detached from the material surface, whether the polymer was displaced by an entity under those conditions, or whether an entity became bound to functional groups of the polymer while the polymer remained attached to the surface. Other screening tests can also be performed by those of ordinary skill in the art.

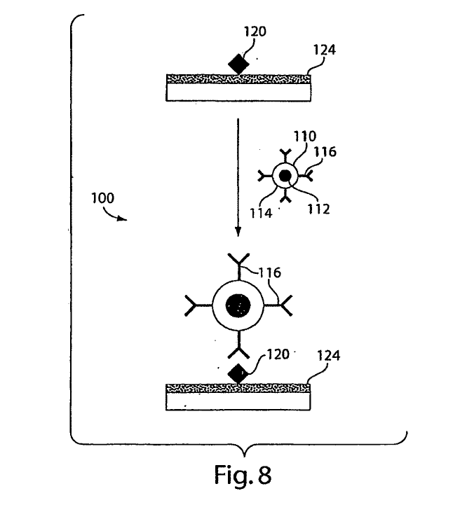

Polymer-coated nanoparticles can be functionalized with binding sites and can be used for analyte detection. For instance, as shown in the embodiment illustrated in FIG. 8, functionalized nanoparticle 110 may include nanoparticle 112 (e.g., a colloidal or semiconductor nanoparticle) and polymer coating 114, which can be functionalized with binding partner 116. Binding partners 116 can interact with analyte 120, which, in some cases, may be attached to a surface 124. In one particular embodiment, polymer coating 114 comprises cysteine-functionalized polyaspartic acid and binding partner 116 includes goat anti-h-IgG. A suitable coupling agent such as 1-ethyl-3-(3-dimethylaminopropyl)carbodiimide (EDC) hydrochloride can be used to attach the antibodies to the polymer-coated nanoparticle. EDC can form a covalent amide bond between a carboxylate group of the polymer and a primary amine group of the antibody.

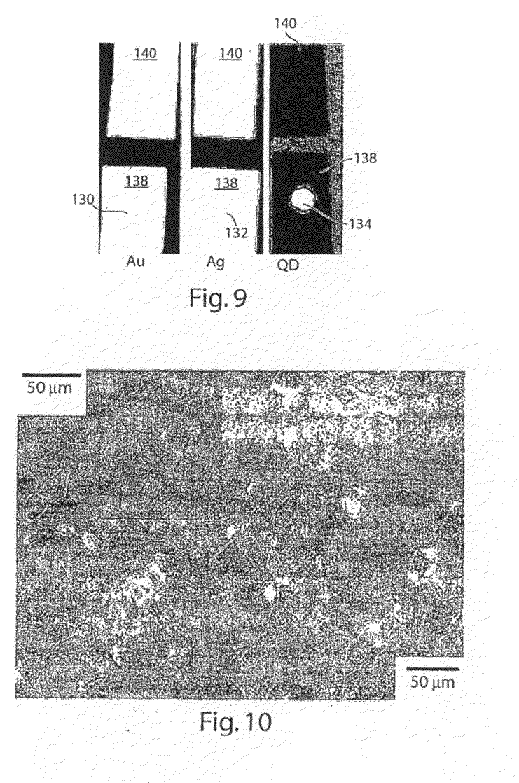

FIG. 9 shows the use of polymer-coated nanoparticles (having a cysteine-functionalized polyaspartic acid coating) conjugated with goat anti-h-IgG to detect IgG from human serum (h-IgG). Gold 130, silver 132, and quantum dot 134 nanoparticles were immobilized onto nitrocellulose membrane strips 138. The strips were immersed in a solution of human serum and the IgG from the serum bound to the anti-h-IgG on the nanoparticles. Strips 140 that did not include immobilized nanoparticles did not allow binding of IgG.

Analytes may be attached to a variety of difference surfaces. The surface may be biological, non biological, organic, inorganic, or a combination of any of these, existing as, for example, a planar or non-planar surface, sheet, slide, wafer, bead, web, fiber, tube, capillary, microfluidic channel, reservoir, strand, precipitate, gel, sphere, container, capillary, pad, slice, film, plate, or other structure. In some cases, the analyte is attached to a surface of a nanoparticle (e.g., a nanotube, nanowire, nanorod, and the like). The surface may have any convenient shape, such as a disc, square, sphere, circle, tube, etc. In some embodiments, the surface is substantially flat but may take on a variety of alternative surface configurations. For example, the surface may have topographies such as raised regions, etched trenches, surface roughness, or the like. Surfaces may also be porous in some embodiments.

In other embodiments, analytes are not attached to a surface (e.g., analytes may be in a solution, suspension, entrapped in a matrix, etc.).

Entities (e.g., antibodies) may be attached (e.g., covalently) to polymer-coated nanoparticles for labeling of components such as cells. For instance, in the embodiment illustrated in FIG. 10, anti-m-EGFR is conjugated with a quantum dot and used to label mouse breast cancer cells.

Nanoparticles described herein may have a variety of shapes, sizes, and/or compositions. For instance, the nanoparticles may be substantially spherical, oval, or rod-like. The nanoparticles may have at least one cross-sectional dimension of less than 100 nm, less than 50 nm, less than 20 nm, less than 10 nm, less than 6 nm, or less than 3 nm. In some cases, the size of the nanoparticle may be measured in combination with a coating of a polymer (e.g., a water-soluble polymer). The polymer-coated nanoparticle may have a cross-sectional dimension of less than 100 nm, less than 50 nm, less than 20 nm, less than 10 nm, less than 6 nm, or less than 3 nm. In some cases, the polymer-coated nanoparticle may have a cross-sectional dimension between 3 and 6 nm, between 4 and 6 nm, or between 4 and 7 nm. Sizes and/or dimensions of nanoparticles may be determined using standard techniques, for example, by measuring the size of a representative number of particles using microscopy techniques (e.g., TEM and DLS).

Nanoparticles may have any suitable material composition. In some embodiments, nanoparticles are colloid particles (e.g., gold, silver, copper, palladium, and/or platinum nanoparticles). In other embodiments, nanoparticles are formed of a magnetic material (e.g., iron oxide). Nanoparticles may also have other material compositions such as zinc oxide, manganese oxide, tin oxide, nickel oxide, chromium oxide, and rare earth metals such as gadolinium chloride, europium, and terbium, etc. Such materials may be chosen depending on, for example, characteristics of the nanoparticle material and/or the ability of a polymer to attach to a surface of the nanoparticle.

In further embodiments, nanoparticles have a composition including one or more semiconductor materials to form “semiconductor nanocrystals” or “quantum dots”. For example, a nanoparticle may be comprised of one or more elements selected from Groups 2, 7, 8, 9, 10, 11, 12, 13, 14, 15, and 16 of the Periodic Table of Elements. These Groups are defined according to IUPAC-accepted nomenclature as is known to those of ordinary skill in the art. In some cases, a nanoparticle may be at least partially comprised of Group 12-16 compounds such as semiconductors. The semiconductor materials may be, for example, a Group 12-16 compound, a Group 13-14 compound, or a Group 14 element. Suitable elements from Group 12 of the Periodic Table of Elements may include zinc, cadmium, or mercury. Suitable elements from Group 13 may include, for example, gallium or indium. Elements from Group 14 that may be used in semiconductor nanoparticles may include, e.g., silicon, germanium, or lead. Suitable elements from Group 15 that may be used in semiconductor materials may include, for example, nitrogen, phosphorous, arsenic, or antimony. Appropriate elements from Group 16 may include, e.g., sulfur, selenium, or tellurium.

In some embodiments, nanoparticles may be binary, tertiary, or higher-alloyed nanocrystals.

Examples of binary semiconductor nanocrystals include, but are not limited to, MgO, MgS, MgSe, MgTe, CaS, CaSe, CaTe, SrS, SrSe, SrTe, BaS, BaSe, BaTe, ZnO, ZnS, ZnSe, ZnTe, CdO, CdS, CdSe, CdTe, HgO, HgS, HgSe, HgTe, AlN, AlP, AlAs, AlSb, Al₂S₃, Al₂Se₃, Al₂Te₃, Ga₂S₃, Ga₂Se₃, GaTe, In₂S₃, In₂Se₃, InTe, SnS, SnSe, SnTe, PbS, PbSe, PbTe, AlP, AlAs, AlSb, GaN, GaP, GaAs, GaSb, InN, InP, InAs, InSb, TiN, TiP, TiAs and TiSb. The specific composition may be selected, in part, to provide the desired optical properties.

Ternary or higher alloyed nanocrystal may have compositions comprising alloys or mixtures of the materials listed above. Ternary alloyed nanocrystals may have a general formula of A¹ₓA²₁₋ₓM, A¹₁₋ₓA²ₓM, A¹₁₋ₓMA²ₓ; or A¹₁₋ₓMA²ₓ; quaternary alloyed nanocrystals may have a general formula of A¹ₓA²₁₋ₓM¹_{y}M²_{1-y}, A¹₁₋ₓA²ₓM¹_{y}M²_{1-y}, A¹ₓA²₁₋ₓM¹_{1-y}M²_{y}, or A¹₁₋ₓA²ₓM¹_{1-y}M²_{y}, where the index x can have a value between 0.0001 and 0.999, between of 0.01 and 0.99, between 0.05 and 0.95, or between 0.1 and 0.9. In some cases, x can have a value between about 0.2, about 0.3, or about 0.4, to about 0.7, about 0.8 or about 0.9. In some particular embodiments, x can have a value between 0.01 and 0.1 or between 0.05 and 0.2. The index y may have a value between 0.001 and 0.999, between 0.01 and 0.99, between 0.05 and 0.95, between 0.1 and 0.9, or between about 0.2 and about 0.8. Identities of A and M in this context will be understood from the exemplary list of species which follows, and other disclosure herein. In some embodiments, A and M can be selected from Groups 2, 7, 8, 9, 10, 11, 12, 13, 14, 15, or 16 of the Periodic Table of Elements. For instance, in some particular embodiments, A¹ and/or A² can be selected from Groups 2, 7, 8, 9, 10, 11, 12, 13 and/or 14, e.g., while M (e.g., M¹ and/or M²) are selected from Groups 15 and/or 16 of the Periodic Table of Elements.

Non-limiting examples of ternary alloyed nanocrystals include ZnSSe, ZnSeTe, ZnSTe, CdSSe, CdSeTe, CdSTe, HgSSe, HgSeTe, HgSTe, ZnCdS, ZnCdSe, ZnCdTe, ZnHgS, ZnHgSe, ZnHgTe, CdHgS, CdHgSe, CdHgTe, ZnPbS, ZnPbSe, ZnPbTe, CdPbS, CdPbSe, CdPbTe, AlGaAs, InGaAs, InGaP, and AlGaAs. Non-limiting examples of quaternary nanocrystal alloys include ZnCdSSe, ZnHgSSe, ZnCdSeTe, ZnHgSeTe, CdHgSSe, or CdHgSeTe, ZnCdSeTe, ZnCdSeS, HgCdSeS, HgCdSeTe, GaInpAs, AlGaAsP, InGaAlP, and InGaAsP. These nanocrystals can have an appropriate bandgap by adjusting the ratio of the precursors used. The ternary or higher alloyed nanocrystals can be used as-is, or they may act as precursors for preparation of higher alloyed nanocrystal structures.

The emission wavelength of a nanoparticle may be governed by factors such as the size and/or composition of the nanoparticle. As such, these emissions may be controlled by varying the particle size and/or composition of the nanoparticle.

The electromagnetic radiation emitted by a nanoparticle may have very narrow bandwidths, for example, spanning less than about 100 nm, preferably less than about 80 nm, more preferably less than about 60 nm, more preferably less than about 50 nm, more preferably less than about 40 nm, more preferably less than about 30 nm, more preferably less than about 20 nm, and more preferably less than 15 nm. In some cases, the electromagnetic radiation emitted by a nanoparticle may have narrow wavelengths, such as between 10 and 20 nm, between 20 and 25 nm, between 25 and 30 nm, between 30 and 35 nm, or between 28 and 32 nm.

The nanoparticle may emit a characteristic emission spectrum which can be observed and measured, for example, spectroscopically. Thus, in certain cases, many different nanoparticles may be used simultaneously, without significant overlap of the emitted signals. The emission spectra of a nanoparticle may be symmetric or nearly so. Unlike some fluorescent molecules, the excitation wavelength of the nanoparticle may have a broad range of frequencies. Thus, a single excitation wavelength, for example, a wavelength corresponding to the “blue” region or the “purple” region of the visible spectrum, may be used to simultaneously excite a population of nanoparticles, each of which may have a different emission wavelength. Multiple signals, corresponding to, for example, multiple chemical or biological assays, may thus be simultaneously detected and recorded.

The following examples are intended to illustrate certain embodiments of the present invention, but are not to be construed as limiting and do not exemplify the full scope of the invention.

### Examples

This example shows a method for synthesizing a polyaspartic acid-based polypeptide functionalized with cysteine and/or methionine, and the coating of nanoparticles using the modified polypeptide. This example also shows that the resulting nanoparticles can be modified with antibodies and used for protein detection and/or cell labeling.

General. All chemicals were purchased from Aldrich, and used as received without further purification. Absorption spectra were obtained with an Agilent 8453 spectrophotometer using a 1-cm path width quartz cell. Fluorescence emission spectra were collected with a Fluorolog FL 3-11 fluorometer using a 1-cm path width quartz cell. An FEI Technai G high-resolution transmission electron microscope was employed for TEM studies. Samples were prepared by placing a drop of an aqueous sample on the carbon-coated copper grid, followed by air drying for 24 h. NMR spectra were recorded on a Bruker 400 MHz NMR spectrometer. h-IgG, anti-h-IgG produced in goat, and bovine serum albumin (BSA) were purchased from Sigma. Anti-m-EGFR produced in goat was purchased from R&D Systems.

Synthesis of Polysuccinimide. Polysuccinimide was synthesized as follows. L-aspartic acid (10 g) was mixed thoroughly with orthophosphoric acid (1 g, 10% by weight of the monomer), and the solid was heated in an oil bath at 180-200° C. for 30 min under argon. The light yellow solid was grinded to a fine powder in a mortar-and-pestle, heated at 200° C. for 6 h, and cooled to room temperature. Water was added, and the sample was filtered through a sintered funnel and washed several times with water until the filtrate was neutral to methanol. The light yellow solid obtained was dried under vacuum overnight to obtain polysuccinimide as an off-white powder.

Nucleophilic Opening of Polysuccinimide with L-Cysteine/Methionine. Polysuccinimide and methyl-protected L-cysteine/methionine was mixed at a molar ratio of 1:1, and dimethylformamide (DMF) was added. The mixture was heated at 50° C. overnight. The thick solution obtained was treated with aqueous NaOH solution (1 N), and stirred for 1 h at room temperature. The reaction mixture was added to methanol dropwise, and the precipitate formed was filtered, washed and dried.

The resulting polymer had multiple thiol and carboxyl groups, and was highly water-soluble. It had an average molecular weight of 10-15 kDa, as determined by gel permeation chromatography (GPC) analysis. The proton nuclear magnetic resonance (NMR) spectrum of the polymer showed characteristic peaks of cysteine/methionine associated with the polymer.

Synthesis of Polymer-Coated Au, Ag and QD Nanoparticles by Ligand Exchange. Near-monodisperse Au and Ag nanoparticles of 2-10 nm were synthesized in toluene in the presence of long-chain fatty acid/fatty amine surfactants as ligands. ZnS-capped CdSe QDs of different colors (corresponding to 2-6 nm in size) were synthesized using octadecene as the high boiling solvent, and fatty amines, trioctylphosphine oxide (TOPO) and trioctylphosphine (TOP) as ligands. As synthesized nanoparticles were purified by ethanol precipitations, and washed with toluene-ethanol. Next, 10-30 mg of purified nanoparticles were dispersed in 10 mL of a reverse microemulsion, which was prepared by mixing 1 mL of Igepal CO-520 with 9 mL of cyclohexane. 100 μL of polymer solution (100 mg/mL of water) were then added and mixed. In the case of QDs, 100 μL of tetramethyl ammonium hydroxide (0.1 M solution in methanol) were added to induce the ligand exchange. After 5 min of vortexing, 1-2 mL of ethanol was added to disrupt the reverse microemulsion, and the polymer-coated to nanoparticles were collected by centrifuging. The precipitate was washed in ethanol for 2-3 more times, and then dissolved in water or a buffer solution. The buffer solution of the polymer-stabilized nanoparticles was stable for at least several months. NMR spectra of the ligand-exchanged nanoparticles confirmed the presence of polymer. Broadening of the proton NMR spectra was observed, suggesting the polymer was adsorbed to the nanoparticle surface.

Ligand-Exchange Method for Aqueous Au and Ag Nanoparticles. Au and Ag nanoparticles of 5-100 nm were synthesized by citrate reduction method or by seeding growth method in the presence of surfactants. After removing the excess surfactants by centrifuging, the nanoparticles were solubilized in distilled water. Typically, 1.0 mL of particle solution (with 1 mM of Au or Ag) was prepared, mixed with 100 μL of polymer solution (100 mg/mL of water), and sonicated for 5 min. After 1 h of incubation, the particle solution was centrifuged to remove any free polymers. The precipitated particles were then dissolved in a buffer solution.

Spectroscopic studies and transmission electron microscopy (TEM) was performed before and after ligand-exchange. No appreciable changes were observed in the particle size, indicating that the polymer effectively capped the nanoparticles and restricted the particle growth upon ligand-exchange. The ligand-exchange scheme was applied to particles of different sizes, so that polymer-stabilized Au and Ag nanoparticles of 2-100 nm and QDs of different emission colors were systematically derived.

Synthesis of Polymer-Coated Au and Ag Nanoparticles in Water. Au and Ag nanoparticles could also be synthesized by direct reduction of the respective metal salts in the presence of polymers. 10 mL of an aqueous solution of gold chloride or silver nitrate (1-10 mM) were prepared and mixed with the aqueous polymer solution (1-100 mg/mL). 2-3 equivalents of freshly prepared aqueous sodium borohydride solution was then injected with rapid stirring. After 2 min, the stirring was stopped, and the solution was diluted if necessary for spectroscopic and other analyses. Particles of 2-5 nm were formed by varying the polymer concentration (FIGS. 3-5).

Colloidal stability of the polymer-stabilized nanoparticles was tested in various buffers, at different ranges of pH, and in presence of salts. The nanoparticle solutions were stable for at least several months without any sign of aggregation and precipitation. Fluorescence stability of polymer-stabilized QD was examined at pHs ranging from 7 to 10. No fluorescence quenching was observed upon ligand-exchange and after several months of preservation in buffer solutions. Depending on the QD size, quantum yields of 10-20% were achieved.

Antibody Conjugation. Polymer-functionalized nanoparticle or QD solution was prepared in aqueous borate buffer (0.02 M) of pH 7.0. The particle concentration was adjusted using UV-visible spectrophotometer to yield a maximum absorbance of 0.2-0.5 for Au, Ag and QD solutions. The common coupling reagent, 1-ethyl-3-(3-dimethylaminopropyl)carbodiimide (EDC) hydrochloride was employed to conjugate antibodies to the nanoparticle surface; 3-4 mg of EDC and 5-6 mg of N-hydroxy succinimide (NHS), dissolved separately in 1 mL of borate buffer, were added to the particle solution. EDC formed a covalent amide bond between the polymer's carboxylate group and the antibody's primary amine group. After 10 min, free reagents were separated using a Sephadex-G25 column, and the particle solution 1 mL) was immediately mixed with 100 μL of antibody (Ab) solution (1 mg of Ab/mL in borate buffer) and incubated for 2-3 h at 4° C. Next, antibody-bound particles were purified from free Ab and excess reagents by centrifuging at 25000 rpm for 5 min. Finally, the precipitated particles were dissolved in 500 μL of 10 mM Tris buffer of pH 7.0 and kept 4° C.

Protein Detection. 1.0 μL of h-IgG solution (1 μg/mL) was spotted on the dry nitrocellulose strip. The strip was then incubated in a blocking buffer solution (containing 0.5% of BSA, 0.5% of Tween 80 and 10 mM of Tris-HCl of pH 7.0) for 1 h. Goat anti-h-IgG was used to conjugate with gold, silver and QD nanoparticles to detect IgG from human serum (h-IgG) after immobilization onto nitrocellulose membrane strips (FIG. 9). This was done by incubating the strips with anti-h-IgG-conjugated nanoparticle solution for 2 h. Next, the strips were washed with Tris buffer solution of pH 7.0 containing 0.5% of Tween 80.

Cell Labeling. Anti-m-EGFR was conjugated with QDs to label mouse breast cancer cells (FIG. 10). High-speed centrifuge and size-exclusion chromatography were used in the purification steps. No particle aggregation or growth was observed during the entire bioconjugation process, indicating that the polymer protection was very effective.

Mouse breast cancer cells were subcultured in 6-well plates using 500 μL of media, followed by overnight incubation at 40° C. for cell attachment on the well plate surface. Next, 20 μL of anti-m-EGFR-conjugated QD solution were added and mixed with the cell culture medium. After 2 h of incubation at 40° C., cells were washed with buffer solution, and the cell culture medium was added. Cells were then observed under fluorescence microscope (Olympus microscope IX71 with DP70 camera) with blue excitation.

Unlike other types of coating that often induced high non-specific interactions during cell labeling, negligible non-specific interaction was observed with the polymer-coated nanoparticles (see inset of FIG. 10). Such low non-specific interaction may be attributed to the finer overall particle size and the negative surface charge achieved with the polymer coating.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A coated nanoparticle comprising:
a nanoparticle comprising a colloidal or semiconductor material; and
a polymer coating on at least a portion of a surface of the nanoparticle, the polymer coating comprising a polypeptide backbone functionalized with amino acid side groups.

2. A coated nanoparticle as in claim 1, wherein the polymer coating comprises a polymer including functional groups selected to attach the coating to the nanoparticle surface and functional groups selected to participate in covalent attachment of a chemical or biological entity to the coating, wherein the polymer is selected such that the coating resists separation from the nanoparticle under conditions of covalent attachment of the chemical or biological entity to the coating.

3. A coated nanoparticle as in claim 1, wherein the polymer coating comprises a backbone that is charged.

4. A coated nanoparticle as in claim 1, wherein the polymer coating comprises a backbone that is negatively charged.

5. A coated nanoparticle as in claim 1, wherein the polymer coating comprises a polyaspartic acid backbone.

6. A coated nanoparticle as in claim 1, wherein the polymer coating comprises a polyglutamic acid backbone.

7. A coated nanoparticle as in claim 1, wherein the polymer is functionalized with cysteine and/or methionine side groups or derivatives thereof.

8. A coated nanoparticle as in claim 1, wherein at least a portion of the amino acid side groups comprises a thiol.

9. A coated nanoparticle as in claim 1, wherein the polymer has a molecular weight of from about 10 kDa to about 20 kDa.

10. A coated nanoparticle as in claim 1, wherein the polymer comprises a chemical or biological entity covalently attached to the polymer.

11. A coated nanoparticle as in claim 1, wherein the chemical or biological entity is a binding partner for a complementary chemical or biological entity.

12. A coated nanoparticle as in claim 1, wherein the nanoparticle comprises a colloidal material.

13. A coated nanoparticle as in claim 12, wherein the colloidal material is Au or Ag.

14. A coated nanoparticle as in claim 1, wherein the nanoparticle comprises a semiconductor material.

15. A coated nanoparticle as in claim 1, wherein the nanoparticle is a quantum dot.

16. A coated nanoparticle as in claim 1, wherein the nanoparticle is formed of a magnetic material.

17. A coated nanoparticle as in claim 1, wherein the nanoparticle comprises zinc.

18. A coated nanoparticle as in claim 1 having a size of less than or equal to 10 nm.

19. A coated nanoparticle as in claim 1 having a size of less than or equal to 5 nm.

20. A polymer comprising a polypeptide backbone functionalized with amino acid side groups that can bind to a surface of a nanoparticle, and that can participate in covalent attachment of a chemical or biological entity to the polymer, present in a sufficient quantity such that when the polymer is applied to a nanoparticle, at least a portion of the nanoparticle surface is coated with the polymer so as to form a single, isolated polymer-coated nanoparticle having a size of less than or equal to 10 nanometers, presenting for attachment functional groups able to participate in covalent attachment of a chemical or biological entity,
wherein the polymer has a molecular weight of from about 10 kDa to about 20 kDa.

21. A polymer as in claim 20, wherein the backbone is charged.

22. A polymer as in claim 20, wherein the backbone is negatively charged. negatively charged.

23. A polymer as in claim 20, wherein the backbone comprises polyaspartic acid.

24. A polymer as in claim 20, wherein at least a portion of the amino acid side groups comprises cysteine or a derivative thereof.

25. A polymer as in claim 20, wherein at least a portion of the amino acid side groups comprises a thiol.

26. A method of forming a polymer-coated nanoparticle comprising:
selecting a nanoparticle and a polymer comprising a polypeptide backbone functionalized with amino acid side groups, the polymer comprising functional groups that can bind to a surface of the nanoparticle; and
coating at least a portion of the nanoparticle surface with the polymer so as to form single, isolated polymer-coated nanoparticle having a size of less than or equal to 10 nanometers.

27. A method as in claim 26, wherein coating at least a portion of the nanoparticle surface with the polymer comprises introducing the nanoparticle to an aqueous in nonaqueous emulsion.

28. A method as in claim 26, wherein coating at least a portion of the nanoparticle surface with the polymer comprises contacting the nanoparticle with a surfactant.
